(19) United States
(12) Patent Application Publication
(10) Pub. No.: US 2020/0096446 A1
(43) Pub. Date: Mar. 26, 2020

(54) MATERIALS AND METHODS TO IMPROVE ACCURACY OF ASSAYS

(71) Applicant: University of Florida Research Foundation, Inc., Gainesville, FL (US)

(72) Inventor: Daniel J. Gibson, Gainesville, FL (US)

(21) Appl. No.: 16/696,002

(22) Filed: Nov. 26, 2019

Related U.S. Application Data
(63) Continuation-in-part of application No. 15/549,168, filed on Aug. 6, 2017, now abandoned.
(60) Provisional application No. 62/117,468, filed on Feb. 18, 2015.

(51) Int. Cl. G01N 21/64; G01N 33/543

(52) U.S. Cl. CPC G01N 21/6428; G01N 33/54306

(57) ABSTRACT

Processes which require the mixing of solutions in definite proportions typically require precise equipment to measure and dispense the solutions to maintain the proportions within a tolerable range. Some processes are not amenable to precise collection, transportation, or mixing of solutions and would therefore cause the proportions to be uncontrolled. Disclosed herein are systems, devices, and methods that allow imprecise volumes of solutions to be utilized while maintaining control of the system.

### CROSS-REFERENCE TO RELATED APPLICATIONS

This application is a continuation-in-part of application Ser. No. 15/549,168, filed Aug. 6, 2017, which was the National Stage of International Application No. PCT/US2016/018375, filed Feb. 18, 2016, which claims benefit of U.S. Provisional Application No. 62/117,468, filed Feb. 18, 2015, which is hereby incorporated herein by reference in its entirety.

### BACKGROUND

Analytical assays, for example, biological assays (bioassays), are sensitive to variations in the sample volume put into the assay reaction. Many medically relevant assays use imprecise methods of collecting biofluid samples. This imprecision paired with the sensitivity to variations in input volume lead to errors in biological assays.

### SUMMARY

Processes which require the mixing of solutions in definite proportions typically require precise equipment to measure and dispense the solutions to maintain the proportions within a tolerable range. Some processes are not amenable to precise collection, transportation, or mixing of solutions and would therefore cause the proportions to be uncontrolled. Disclosed herein are systems, devices, and methods that allow imprecise volumes of solutions to be utilized while maintaining control of the system.

The disclosed system involves at least two fluorescent signal generating molecules. One fluorescent signal generating molecule measures the amount of fluid input into the system. The other fluorescent signal generating molecule(s) transduce the presence and/or activity of molecules within the solution. The accuracy of the measured presence or activity of molecules is dependent upon the accuracy of the input/sample volume. The fluorescent signal generated by the volume detection molecule is used to 1) determine that an adequate amount of sample has been added, 2) that the amount added is not too much, and 3) to adjust the interpreted quantity of the presence or activity of molecules according to the amount of volume input.

The “starting solution” contains a pre-determined and controlled amount of a “volume control molecule”. Imprecisely added amounts of “sample solution” (i.e. containing “assay molecule”) then dilute the volume control molecule in proportion to the amount of volume input. If too much sample solution is added, this dilutes the control molecule beyond a threshold, which can raise a flag and cause a failure state. The flag could provide a message stating that an assay failed due to too much sample added. Alternatively, the flag could cause more starting solution to be added to compensate. If too little sample solution is added, the control molecule is insufficiently diluted, which can raise a flag and cause a failure state. The flag could provide a message stating that an assay failed due to insufficient sample added. Alternatively, the flag could cause more sample solution to be added. If a scalable amount is added, this can cause a proportional response. The proportional response could be the adjustment of calibration constants; such as in a molecular biological assay.

The disclosed system can therefore involve a reaction vessel containing a starting solution and a detection device. The starting solution can contain a first fluorophore and a second fluorophore that emits at a different wavelength than the first fluorophore. For example, the second fluorophore can be directly or indirectly linked to a detection agent that selectively binds an analyte. In some embodiments, the starting solution is contained in the reaction vessel at a fixed volume within a 0.1% to 5% variance, such as 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% variance from reaction vessel to reaction vessel.

The disclosed detection device is in some embodiments configured to detect fluorescent signals from the first fluorophores and the second fluorophores when a sample is added to the reaction vessel. Therefore, in some embodiments, the device contains a monochromatic light source to excite the first fluorophores and the second fluorophores to produce a first fluorescent signal and a second fluorescent signal, respectively. For example, the monochromatic light source can be a laser, a band-emitting LED, a filtered broad spectrum light source, or a grating controlled light dispersion system.

The device can also contain a wavelength or band-width sensitive light sensor to detect and convert the first fluorescent signal and the second fluorescent signal into electrical signals. For example, the wavelength or band-width sensitive light sensor can be a photodiode, LED, photoresistor, photomultiplier tube, CCD array, or CMOS array.

The device can also contain a processor programmed by software or firmware to determine if the first fluorescent signal is within a pre-determined range as a pre-condition for displaying the results of the second fluorescent signal. In some embodiments, the processor is programmed to cause a failure state if the first fluorescent signal is below the pre-determined range as an indication that too much of the sample solution added. In some embodiments, the processor is programmed to cause a failure state if the first fluorescent signal is above the pre-determined range as an indication that an insufficient amount of the sample solution was added.

The disclosed systems and devices can be used with any agent suitable for detecting an analyte that can be linked directly or indirectly with a fluorophore. All types of biomolecules can be adapted for use as detection agents, depending on the analyte. For example, the detection agent can be a polypeptide, protein, carbohydrate, or polynucleotide that binds or metabolizes the analyte. The detection agent can therefore be a small peptide or large macromolecule (protein). Examples include natural and synthetic ligands, receptors, antibodies, and aptamers. Suitable polynucleotides include oligonucleotide aptamers.

Therefore, in some embodiments, the detection agent is an antibody, soluble receptor, oligonucleotide, or aptamer. For example, the second fluorophore can be conjugated to a primary antibody specific for the analyte or a secondary antibody that binds a primary antibody specific for the analyte. As another example, the second fluorophore can be conjugated to a polypeptide or polynucleotide that specifically binds or metabolizes the analyte.

Also disclosed herein is a method for detecting an analyte that involves providing a reaction vessel containing a fixed volume of a starting solution containing a first fluorophore and a detection agent that selectively binds an analyte that is directly or indirectly linked to a second fluorophore that emits at a different wavelength than the first fluorophore. In some embodiments, the starting solution is contained in the reaction vessel at a fixed volume within a 0.1% to 10% variance, including 0.1% to 5% variance, 1% to 10% variance, and 1% to 5% variance, which includes 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% variance, from reaction vessel to reaction vessel. In some embodiments, the method involves measuring the volume of the starting solution to confirm this variance. In some embodiments, the processor is programmed to cause a failure state if the starting solution is outside of the selected variance.

The method then involves adding an imprecise volume of a sample solution comprising the analyte to the reaction vessel to produce a reaction mixture. This reaction mixture is then analyzed with a detection device as described above.

In some embodiments, the processor is programmed to cause a failure state if the first fluorescent signal is below the pre-determined range as an indication that too much of the sample solution added. In these embodiments, the method can further involve adding additional starting solution to the reaction vessel and repeating the analyzing step.

In some embodiments, the processor is programmed to cause a failure state if the first fluorescent signal is above the pre-determined range as an indication that an insufficient amount of the sample solution was added. In these embodiments, the method can further involve adding additional sample solution to the reaction vessel and repeating the analyzing step.

The disclosed methods can be used with any form of analyte that can be detected in a solution with a fluorophore. In some embodiments, the sample solution is a biofluid sample from a subject and the analyte is a clinically relevant biomolecule. For example, in some embodiments, the biofluid sample is exhaled breath, whole blood, blood plasma, blood serum, urine, tears, semen, saliva, buccal mucosa, interstitial fluid, lymph fluid, meningeal fluid, amniotic fluid, glandular fluid, sputum, feces, perspiration, mucous, vaginal secretion, cerebrospinal fluid, wound exudate, wound homogenate, wound fluid, aqueous humor, vitreous humor, bile, endolymph, perilymph, pericardial fluid, pleural fluid, or synovial fluid. In some embodiments, the biofluid sample is an extract of a tissue selected from brain, eyes, pineal gland, pituitary gland, thyroid gland, parathyroid glands, thorax, heart, lungs, esophagus, thymus gland, pleura, adrenal glands, appendix, gall bladder, urinary bladder, large intestine, small intestine, kidneys, liver, pancreas, spleen, stoma, prostate gland, testes, ovaries, or uterus.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows mixing of an assay reaction mixture with a biofluid sample producing dilution of the solute present in the assay reaction mixture.

FIG. 2 shows examples of biofluid samples of insufficient volumes mixed with an assay reaction mixture.

FIG. 3 shows an example of a biofluid sample of excessive volume mixed with an assay reaction mixture.

FIG. 4 shows an example of biofluid sample mixed with an assay reaction mixture, wherein the biofluid sample has a volume that is nominally different from the desired input volume.

FIG. 5 shows an example of corrected standard curve adjusted based on the volume of the standard solution added in the assay reaction mixture.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The disclosed system involves at least two fluorescent signal generating molecules. One fluorescent signal generating molecule measures the amount of fluid input into the system. The other fluorescent signal generating molecule(s) transduce the presence and/or activity of molecules within the solution. The accuracy of the measured presence or activity of molecules is dependent upon the accuracy of the input volume. The fluorescent signal generated by the volume detection molecule is used to 1) determine that an adequate amount of sample has been added, 2) that the amount added is not too much, and 3) to adjust the interpreted quantity of the presence or activity of molecules according to the amount of volume input.

The “starting solution” contains a pre-determined and controlled amount of a “volume control molecule”. Imprecisely added amounts of “sample solution” (i.e. containing “assay molecule”) then dilute the volume control molecule in proportion to the amount of volume input. If too much sample solution is added, this dilutes the control molecule beyond a threshold, which can raise a flag and cause a failure state. The flag could provide a message stating that an assay failed due to too much sample added. Alternatively, the flag could cause more starting solution to be added to compensate. If too little sample solution is added, the control molecule is insufficiently diluted, which can raise a flag and cause a failure state. The flag could provide a message stating that an assay failed due to insufficient sample added. Alternatively, the flag could cause more sample solution to be added. If a scalable amount is added, this can cause a proportional response. The proportional response could be the adjustment of calibration constants; such as in a molecular biological assay.

The disclosed system employs fluorescent molecules which are able to provide usable signal in many solution types. Fluorescence also enables “surface” measurement of the concentration as compared to absorbance type measurements which are sensitive to the light path distance through a sample.

In some embodiments, such as biological assay systems, the assay molecule can generate a signal in one fluorescent band (e.g. blue-excited green) while the volume control molecule generates a signal in another, non-overlapping band (e.g. green-excited red, or red-excited infrared). The two signals can be used to adjust the calibration equation and/or constants to account for imprecise volume input as might happen from a clinical sample collected with a swab. The calibration equation and/or constants would be informed based upon empirical testing with the particular fluorescence generating measurement sensor/molecule.

The system can employ monochromatic light source to excite the fluorophores. For example, the source can be a laser “line,” a band-emitting LED, a filtered broad spectrum light source, or a grating controlled light dispersion system.

The system can also employ wavelength or band-width sensitive light sensors to transduce the fluorescent signal into an electrical signal. Any light transducer which is amenable to the application can be used. Examples, include a photodiode, LED, photoresistor, photomultiplier tube, CCD array, or CMOS array.

The signals from the light transducers can be amplified and/or conditioned if needed. The raw, amplified, and/or conditioned signals can be fed into “hardwired” logic circuits. Alternatively, the raw, amplified, and/or conditioned signals can be quantified. This quantification can be analog or digital via a DAC. The digitized signals can be sent to a processor, such as a microprocessor or microcomputer.

The digitized signals can be analyzed, e.g. by pre-programmed firmware and/or software. The outcome can be displayed in raw form as intensities of the fluorescent signals. The outcome can be used to quantify the volume of solution input. The outcome can be used to adjust calibration constants/equations for interpretation. The outcome can be used to signal other systems. The outcome can be used to scale data. The outcome can be displayed on a screen. The outcome can be transmitted to a database

In some embodiments, the disclosed fluorophore is a fluorescent chemical compound that can re-emit light upon light excitation. Usually, the emitted light has a different wavelength than that of the excitation light. Various fluorophores that can be used in the compositions and methods of the current invention are well known to a person of ordinary skill in the art and such embodiments are within the purview of the current invention.

In accordance with the subject invention, an assay can be rejected based on insufficient dilution, i.e., insufficient sample, or based on excessive dilution, i.e., too much sample. The dilutions of the solute within an acceptable range based on predetermined boundaries can be used to calculate a scaling factor that facilitates accurate measurement of the sample volume that nominally deviates from the desired input volume. A person of ordinary skill in the art can modify the assay reaction mixture of the current invention to suit various types of assays and such embodiments are within the purview of the current invention.

Because the disclosed methods can be used in clinically-relevant bioassays, the samples used in the methods of the current invention include biofluids obtained from a subject. Non-limiting examples of biofluids that can be used in the methods of the current invention include exhaled breath, whole blood, blood plasma, blood serum, urine, tears, semen, saliva, buccal mucosa, interstitial fluid, lymph fluid, meningeal fluid, amniotic fluid, glandular fluid, sputum, feces, perspiration, mucous, vaginal secretion, cerebrospinal fluid, wound exudate, wound homogenate, wound fluid, aqueous humor, vitreous humor, bile, endolymph, perilymph, pericardial fluid, pleural fluid, and synovial fluid. The biofluids can be appropriately treated before they are used pursuant to the methods of the current invention. Biofluids also include extracts of a tissue. The tissues can be appropriately treated to produce biofluid for use according to the current invention. A person of ordinary skill in the art can utilize various tissue treatments to produce biofluids.

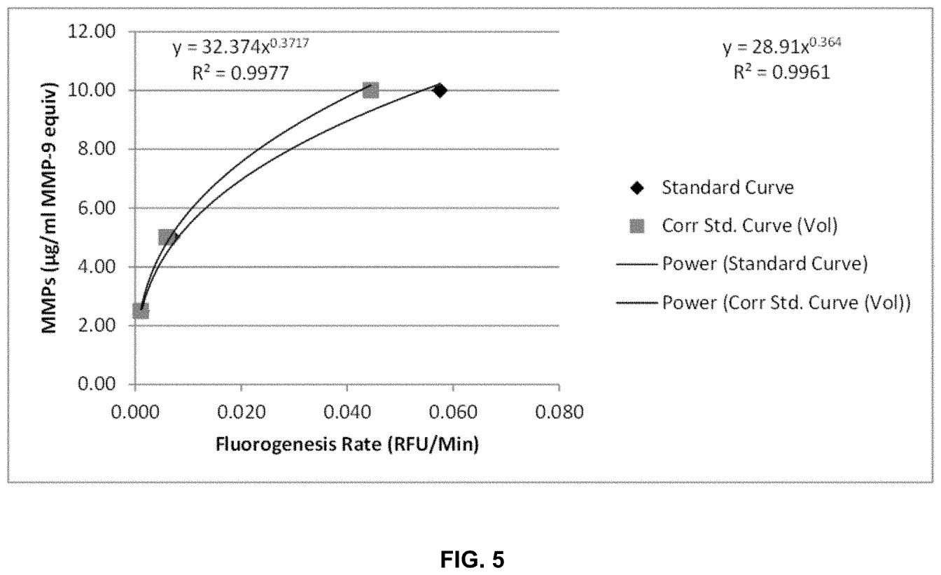

In some embodiments, the sample is a standard solution containing a known concentration of the analyte to be assayed. Such standard solutions are used to produce a standard curve of the analyte. The assay reaction mixture of the current invention allows accurate determination of the standard solution mixed in the set of reactions used to produce the standard curve for the analyte. An example of a standard curve and the corrected standard curve is provided in FIG. 5.

Also disclosed herein are assay kits for detection and/or quantification of an analyte in a sample. The kit can separately provide various constituents of the assay reaction mixture. A user can then mix the contents to produce the assay reaction mixture for use according to the methods of the current invention. In certain embodiment, the assay kits are designed for detection and/or quantification of a clinically relevant biomolecule in a biological sample such as, for example, a biofluid sample, obtained from a subject. Non-limiting examples of the biomolecules that can be assayed according to the kits and methods of the current invention include matrix metalloproteinase (MMP), neutrophil elastase (NE), and nitrogen dioxide (NO2). In certain embodiments, the assay is Fluorescence Resonance Energy Transfer (FRET)-based assay such as, for example, MMP-FRET, human NE-FRET, or NO₂ fluorescence assay.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Determining the Volume of the Sample and Rejecting/Accepting a Test Result

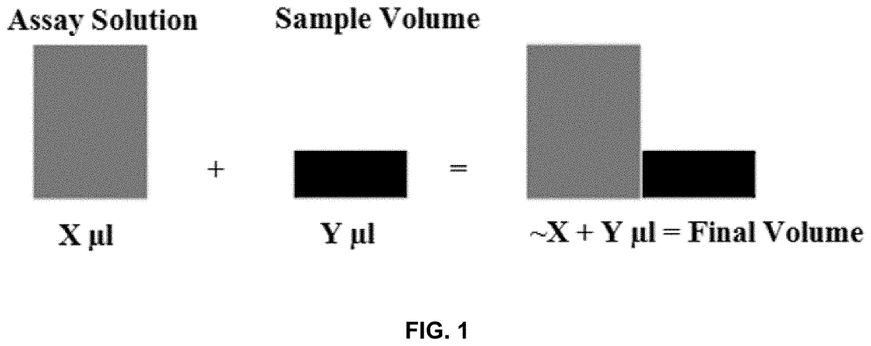

In an assay, 350 μl of assay reaction mixture is mixed with 150 μl of sample to produce 30% v/v dilution of the chemicals, including a solute, within the assay reaction mixture (FIG. 1). The solute is a pigment or a fluorophore that can be measured spectroscopically. The pigment concentration can be measured either by absorption spectroscopy or by fluorescence spectroscopy and the pigment or the fluorophores can be quantified to determine how the anticipated dilution deviates from 30%.

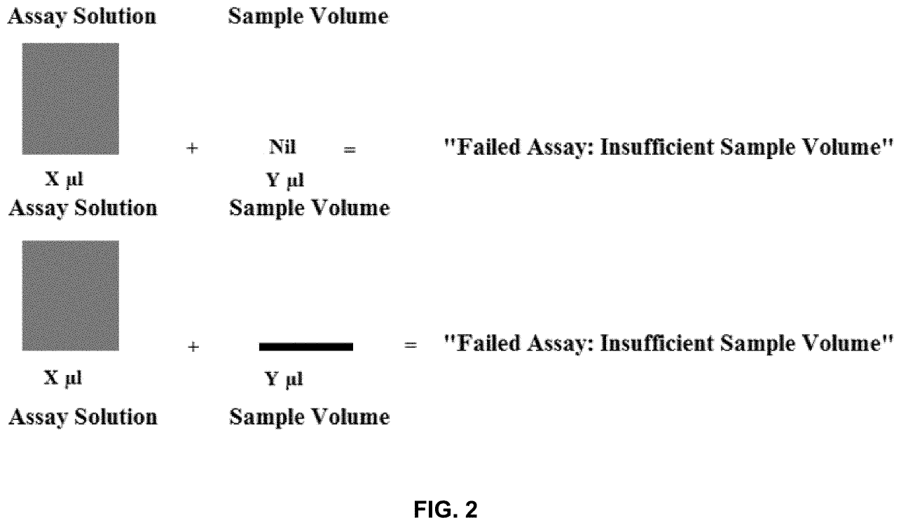

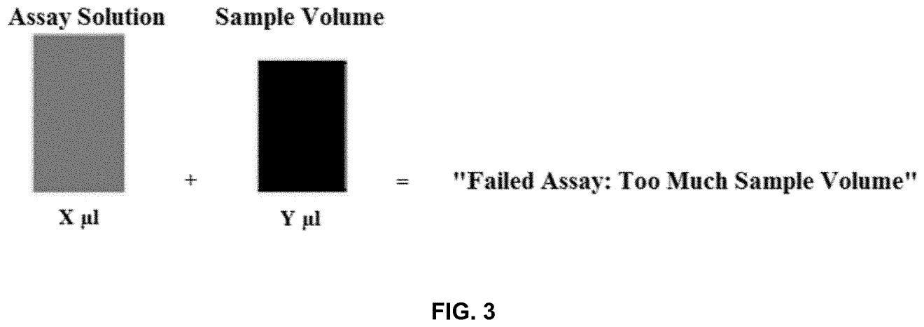

If there is no dilution, or the dilution is below the nominal 30% dilution by a pre-determined level, for example, 10-20% below nominal, then the assay can be rejected as invalid due to insufficient sample volume (FIG. 2). Similarly, if the dilution is above the nominal 30% dilution by a predetermined level, for example, 10-20% above nominal, then the assay can be rejected as invalid due to excessive sample volume (FIG. 3).

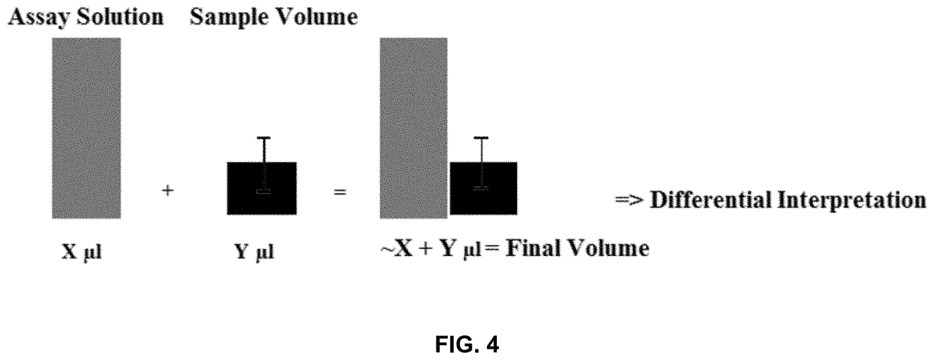

In the event that the dilution is nominally above or below the nominal 30% dilution, e.g., the dilution is within a predetermined dilution range, for example, within ±10-20%, the dilution can be used to establish a scaling factor (FIG. 4). The scaling factor can then be applied to the assay such that a different set of calibration coefficients are used for every sample. Also if the dilution is within the linear range of the assay, a simple scaling of the data can be used to improve the accuracy of the assay.

### Example 2: Determining the Volume of Biofluid Samples in Assays for Clinically Relevant Biomolecules

The compositions and methods of the current invention can be used to determine the volume of biofluid samples used in assays for clinically relevant biomolecules, for example, in medical diagnostic assays. Non-limiting examples of such assays include the assays for matrix MMP-FRET assay, NE-FRET assay, or NO2-FRET assay.

In further embodiments, the current invention is modified to suit bioassays described in, for example, United States Patent Application Publication Nos. 2012/0136054, 2012/0135443, 2012/0122133, 2012/0078162, 2009/0258382, and 2008/0176263, the contents of which are incorporated herein by reference in their entireties.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A system, comprising
a reaction vessel containing a starting solution comprising
a first fluorophore, and
a detection agent that selectively binds an analyte that is directly or indirectly linked to a second fluorophore that emits at a different wavelength than the first fluorophore;
a detection device configured to detect fluorescent signals from the first fluorophores and the second fluorophores when a sample is added to the reaction vessel, wherein the device comprises:
a monochromatic light source to excite the first fluorophores and the second fluorophores to produce a first fluorescent signal and a second fluorescent signal, respectively;
a wavelength or band-width sensitive light sensor to detect and convert the first fluorescent signal and the second fluorescent signal into electrical signals;
a processor programmed by software or firmware to determine if the first fluorescent signal is within a pre-determined range as a pre-condition for displaying the results of the second fluorescent signal.

2. The system of claim 1, wherein the monochromatic light source comprises a laser, a band-emitting LED, a filtered broad spectrum light source, or a grating controlled light dispersion system.

3. The system of claim 1, wherein the wavelength or band-width sensitive light sensor comprises a photodiode, LED, photoresistor, photomultiplier tube, CCD array, or CMOS array.

4. The system of claim 1, wherein the processor is programmed to cause a failure state if the first fluorescent signal is below the pre-determined range as an indication that too much of the sample solution added.

5. The system of claim 1, wherein the processor is programmed to cause a failure state if the first fluorescent signal is above the pre-determined range as an indication that an insufficient amount of the sample solution was added.

6. The system of claim 1, wherein the detection agent is an antibody, soluble receptor, oligonucleotide, or aptamer.

7. The system of claim 6, wherein the second fluorophore is conjugated to a primary antibody specific for the analyte or a secondary antibody that binds a primary antibody specific for the analyte.

8. The system of claim 6, wherein the second fluorophore is conjugated to a polypeptide or polynucleotide that specifically binds or metabolizes the analyte.

9. The system of claim 1, wherein the starting solution is contained in the reaction vessel at a fixed volume within a 1% variance.

10. A method for detecting an analyte, comprising
(a) providing a reaction vessel containing a fixed volume of a starting solution comprising:
(1) a first fluorophore, and
(2) a detection agent that selectively binds an analyte that is directly or indirectly linked to a second fluorophore that emits at a different wavelength than the first fluorophore;
(b) adding an imprecise volume of a sample solution comprising the analyte to the reaction vessel to produce a reaction mixture;
(c) analyzing the reaction mixture with a detection device that comprises:
(1) a monochromatic light source to excite the first fluorophores and the second fluorophores to produce a first fluorescent signal and a second fluorescent signal, respectively;
(2) a wavelength or band-width sensitive light sensor to detect and convert the first fluorescent signal and the second fluorescent signal into electrical signals; and
(3) a processor programmed by software or firmware to determine if the first fluorescent signal is within a pre-determined range and to display the results of the second fluorescent signal if the first fluorescent signal is within the pre-determined rage, or display an error message if the first fluorescent signal is not within the pre-determined rage.

11. The method of claim 10, wherein the monochromatic light source comprises a laser, a band-emitting LED, a filtered broad spectrum light source, or a grating controlled light dispersion system.

12. The method of claim 10, wherein the wavelength or band-width sensitive light sensor comprises a photodiode, LED, photoresistor, photomultiplier tube, CCD array, or CMOS array.

13. The method of claim 10, wherein the processor is programmed to cause a failure state if the first fluorescent signal is below the pre-determined range as an indication that too much of the sample solution added.

14. The method of claim 13, further comprising adding additional starting solution to the reaction vessel and repeating step (c).

15. The method of claim 10, wherein the processor is programmed to cause a failure state if the first fluorescent signal is above the pre-determined range as an indication that an insufficient amount of the sample solution was added.

16. The method of claim 15, further comprising addition additional sample solution to the reaction vessel and repeating step (c).

17. The method of claim 10, wherein the detection agent is an antibody, soluble receptor, oligonucleotide, or aptamer.

18. The method of claim 17, wherein the second fluorophore is conjugated to a primary antibody specific for the analyte or a secondary antibody that binds a primary antibody specific for the analyte.

19. The method of claim 17, wherein the second fluorophore is conjugated to a polypeptide or polynucleotide that specifically binds or metabolizes the analyte.

20. The method of claim 10, wherein the starting solution is contained in the reaction vessel at a fixed volume within a 1% variance.
